(19) United States
(12) Patent Application Publication
(10) Pub. No.: US 2010/0298648 A1
(43) Pub. Date: Nov. 25, 2010

(54) SURGICAL INSTRUMENT

(76) Inventor: Bruce Gray, Bentley (AU)

Correspondence Address: GOTTLIEB RACKMAN & REISMAN PC, 270 MADISON AVENUE, 8TH FLOOR, NEW YORK, NY, 10016-0601, US

(21) Appl. No.: 12/754,666

(22) Filed: Apr. 6, 2010

(30) Foreign Application Priority Data
Apr. 6, 2009 (AU) 2009901485

(51) Int. Cl. A61B 1/32

(52) U.S. Cl. 600/213

(57) ABSTRACT

A surgical instrument (11) comprising a handle (13), a retractor blade (15) and an attachment device (17) to releasably secure the retractor blade to a first end (19) of the handle. The surgical instrument is arranged such that in a first condition the attachment device secures the blade to the handle whereby the blade may rotate relative to the handle, whilst in a second condition the attachment means secures the blade to the handle such that the blade is fixed relative thereto.

### FIELD OF THE INVENTION

The present invention generally relates to a surgical instrument. In particular the invention relates to a surgical instrument for supporting a surgical tool such that the tool may rotate or be fixed relative to a handle.

### BACKGROUND ART

During surgical operations surgeons are often required to support tissue away from the area of operation so they are able to access the internal anatomy of their patient. In order to achieve this surgeon's use one or more retractor tools which are generally supported on a frame.

Retractor tools comprise a retractor blade connected to a handle. Depending upon its application, the surgeon will either use a retractor tool comprising a retractor blade fixed relative to the handle or a retractor blade which is rotatable with respect to the handle. Retractor tools also incorporate a further variation wherein the blade may be removed from the handle or is permanently secured thereto.

During surgery a surgeon will generally require a multiple of fixed and rotatable retractor tools. As such a surgeon is required to have on hand a number of retractor tools comprising both those with fixed blades, and those which are rotatable.

The preceding discussion of the background to the invention is intended only to facilitate an understanding of the present invention. It should be appreciated that the discussion is not an acknowledgment or admission that any of the material referred to was part of the common general knowledge as at the priority date of the application.

### SUMMARY OF THE INVENTION

It is an object of this invention to provide a surgical instrument which will minimise the number of instruments required on hand during an operation.

The present invention provides a surgical instrument comprising a handle and a surgical tool releasably secured thereto, the surgical tool is movable between a first condition whereby the tool is free to rotate relative to the handle, and a second condition whereby it is fixed relative to the handle.

In contrast to the prior art, the present invention provides a single surgical instrument which is able to support a surgical tool such that it may be fixed relative to the handle or may be free to rotate. As a result the present invention will reduce the number of surgical instruments required during surgery.

Throughout the specification the term rotate is considered to include pivot, swivel or any circular motion.

The surgical tool may comprise an attachment end for attaching to a first end of the handle.

The surgical tool may be in the form of a retractor blade.

In one aspect of the invention the first end of the handle has an opening. The opening may extend through the handle. The attachment end of the retractor blade may support a spigot which may be releasably secured in the opening so that the retractor blade is rotationally supported on the handle.

In another aspect of the invention the attachment end of the retractor blade has an opening. The opening may extend through the attachment end. The first end of the handle may support a spigot which may be releasably secured in the opening.

Preferably when the tool is in the second condition, the tool may first be rotated to the desired position before being fixed relative to the handle. The first end of the handle and attachment end of the surgical tool may cooperate when in the second condition such that the surgical tool may be fixed relative to the handle at any one of a series of predetermined angular positions.

The spigot may comprise an annular groove.

The handle may incorporate a detent. The detent may be in the form of a spring loaded projection which is biased outwardly into the opening. The projection may be adapted to be received in the groove when the surgical tool is connected to the handle, whereby the surgical tool is releasably secured to the handle.

The first end of the handle may have a first side adapted to cooperate with the attachment end of the surgical tool such that the surgical tool is fixed relative to the handle as well as being secured thereto. With this arrangement the surgical tool is in the second condition. Preferably the first side of the handle provides a first mating face, whilst the attachment end of the surgical tool provides a complementary second mating face such that when the two mating faces are in mating relationship they are locked relative to each other.

The mating faces may be in the form of one or more projections, slots or similar arrangement whereby the faces may be locked relative to each other. Preferably the mating faces may be indexed such that the surgical tool may be fixed relative to the handle at various angles thereto.

The first end of the handle may have a second side which is relatively smooth, whereby when the first side of the handle is adjacent the second mating face of the surgical tool the surgical tool is free to rotate whilst still being releasably secured to the handle. With this arrangement the surgical tool is in the first condition.

The present invention further provides a surgical instrument comprising a handle, a retractor blade and an attachment device to releasably secure the retractor blade to a first end of the handle, whereby in a first condition the attachment device secures the blade to the handle such that the blade may rotate relative to the handle, whilst in a second condition the attachment means secures the blade to the handle such that the blade is fixed relative thereto.

The attachment device may comprise a spigot which is received in an opening.

In one aspect of the invention the opening is in the first end of the handle, whilst the spigot extends from the retractor blade.

In another aspect of the invention the opening is in the retractor blade whilst the spigot extends from the first end of the handle.

The attachment means may also comprise a detent, retractably extending into the opening, the end of the detent being adapted to be received in an annular groove on the spigot when the spigot is received in the opening. The detent may be in the form of a projection biased outwardly from the handle into the opening.

The attachment device may provide a first mating face and a second mating face, the first and second mating faces being complementary to each other such that they may lock together. The first mating face and second mating face may be in the form of one or more projections, slots or similar arrangements. The first mating face and second mating face may be locked at various angles such that the retractor blade may be locked relative to the handle at various angles.

The first mating face may be provided by the handle. The second mating face may be provided at an attachment end of the retractor blade. The handle may also have a third mating face. The third mating face may be relative smooth such that when the third mating face is adjacent the second mating face the two surfaces cannot lock together, allowing the retractor blade to rotate relative to the handle whilst still being releasably secured thereto.

Whilst mention is made here to a handle, it is to be understood that the scope of this invention is not limited to such a term and may mean support rod, support frame or similar, and may be hand held or connected to a frame or other device.

### BRIEF DESCRIPTION OF THE DRAWINGS

The invention will be better understood by reference to the following description of two embodiments thereof as shown in the accompanying drawings in which:

FIG. 1 is a perspective view of a surgical instrument according to a first embodiment, the surgical instrument is shown attached to a support frame;

FIG. 2 is a side view of a handle of the surgical instrument shown in FIG. 1;

FIG. 3 is a perspective view of a surgical tool of the surgical instrument shown in FIG. 1, in this case a retractor blade;

FIG. 4 is a cross sectional side view of a first end of the handle shown in FIG. 1;

FIG. 5 is a perspective side view of a first end of the handle shown in FIG. 1;

FIG. 6 is a plan view of a first end of the handle shown in FIG. 1;

FIG. 7 is a plan view of a first end of the handle according to a second embodiment of the invention;

FIG. 8 is a cross sectional side view of the first end of the handle shown in FIG. 7; and

FIG. 9 is a plan view of a second mating surface.

### BEST MODE(S) FOR CARRYING OUT THE INVENTION

Surgical instruments of the type comprising a handle for attachment to a support frame, and a surgical tool, such as a retractor blade are commonly used in surgical operations to support tissue away from the area of operation. The below description is directed to an embodiment of the present invention whereby the retractor blade may be releasably secured to a handle whereby in one condition it may rotate relative thereto, whilst in a second condition it may be fixed relative thereto.

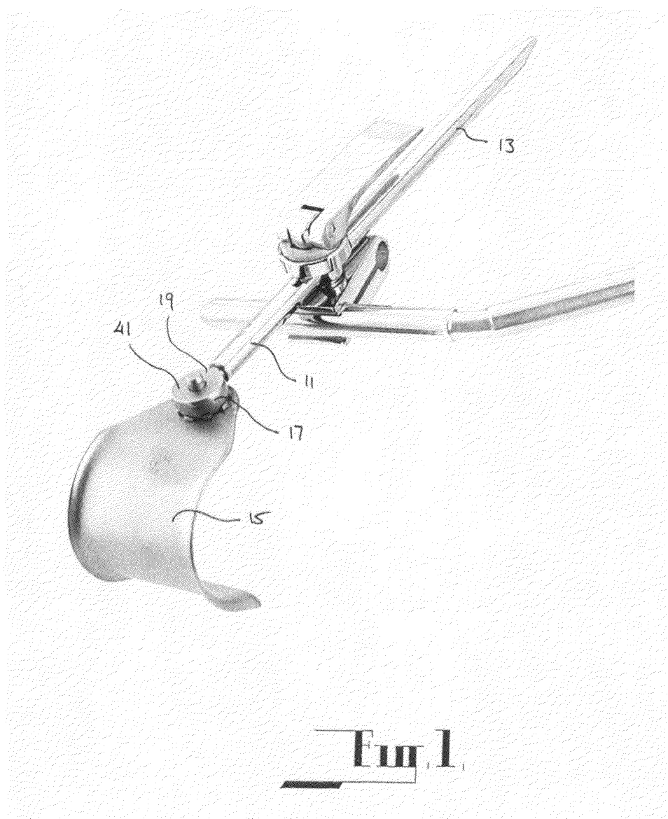

Referring to FIGS. 1 to 3, the invention according to the first embodiment is in the form of a surgical instrument 11 comprising a handle 13 and a surgical tool, in the form of a retractor blade 15. The retractor blade 15 and handle 13 are connected via an attachment device 17.

The attachment device 17 incorporates an opening 21 which, in this embodiment, extends through a first end 19 of the handle 13. The opening 21 is adapted to receive a spigot 23, which in this case extends from an attachment end 25 of the retractor blade 15. The spigot has an annular groove 27, for reasons which will be described below.

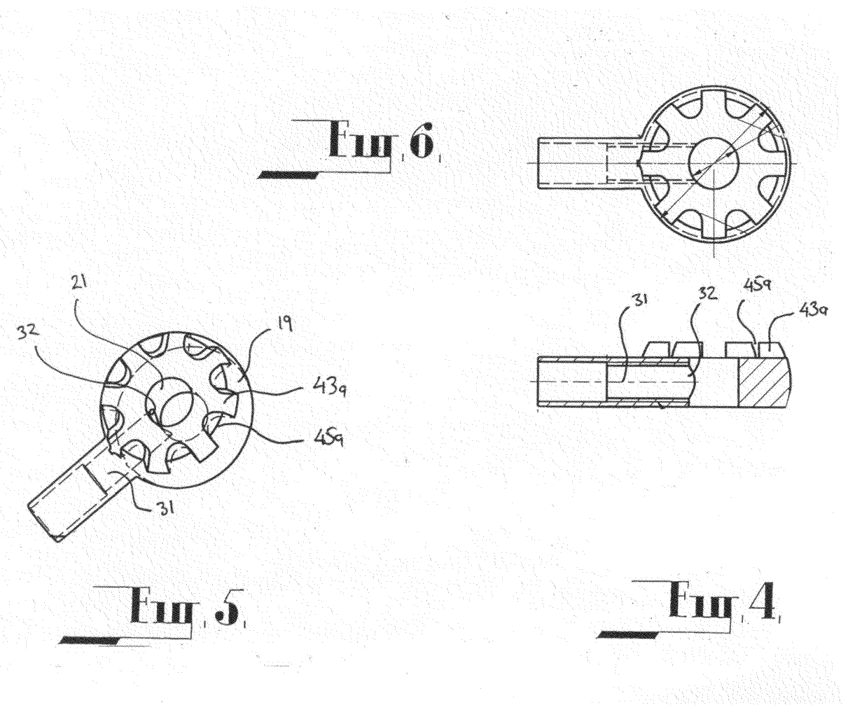

The attachment device 17 also comprises a detent 29 which is housed in the handle. As shown in FIGS. 4 to 6, the detent 29 is located in a recess 31. The recess 31 extends along a portion of the longitudinal axis of the handle 13 and terminates at the opening 21 such that the recess 31 opens into the opening 21. This allows a projection 33 to be received in the recess 31 such that a portion of the projection projects into the opening 21, as can be seen in FIG. 2. The projection 33 is biased outwardly from the recess 31 by a spring plunger (not shown).

When the retractor blade 15 and handle 13 are connected, the projection 33 is received in the groove 27 to releasably secure the retractor blade 15 to the handle 13.

The attachment means 17 also comprises a first mating face 37, a second mating face 39 and a third mating face 41.

As best shown in FIGS. 4 to 6, the first mating face 37 is located on one side of the first end 19 of the handle 13. The first mating face 37 has a plurality of angularly spaced, finger like projections 43a which define a plurality of slots 45a therebetween.

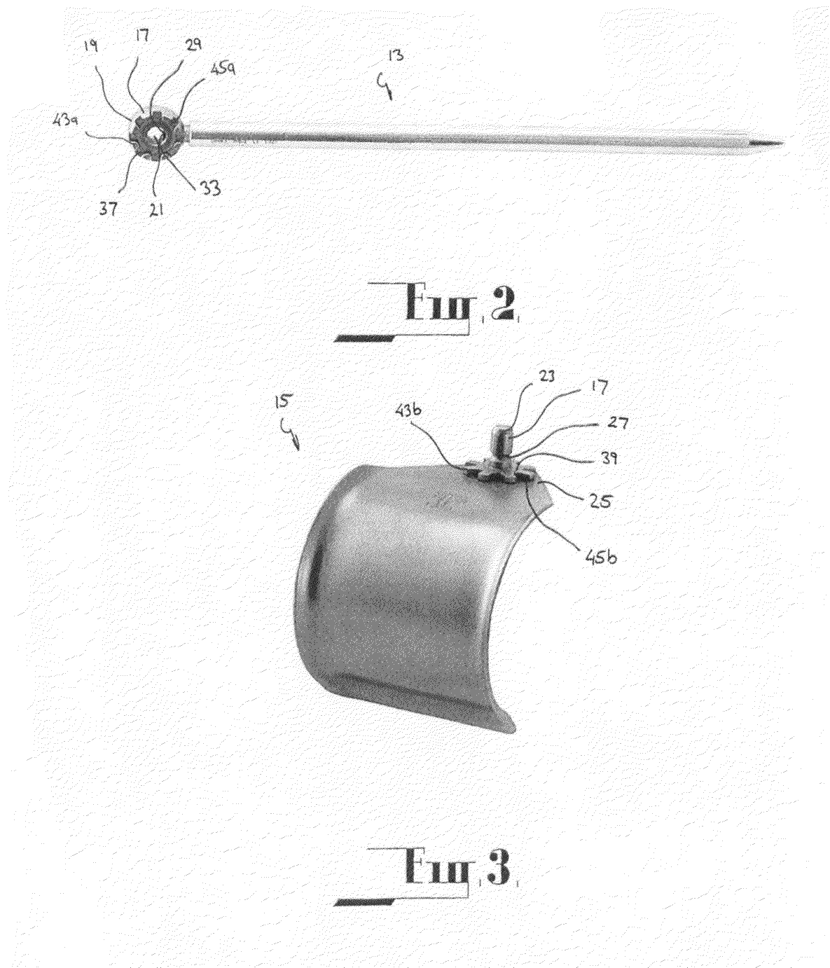

As best shown in FIG. 3, the second mating face 39 comprises similar finger like projections 43b and slots 45b as those of the first mating face 37, but are configured to be complementary to those of the first mating face 37. As a result when the first mating face 37 and second mating face 39 are adjacent the projections 43 in one mating face can be aligned with the slots 45 in the other mating face such that they are received therein to lock the first mating face 37 relative to the second mating face 39, obviously locking the retractor blade 15 relative to the handle 13.

As the first mating face 37 locks with the second mating surface 39, the projection 33 of the detent 29 aligns with the annular groove 27 of the spigot 23 to be received therein. When the projection 33 is received in the groove 27 the retractor blade 15 is releasably secured to the handle 13. When in this position the surgical instrument 11 is in the second condition.

As the finger like projections 43a,b are angularly spaced the handle 13 and retractor blade may be fixed relative to each other at various angles by first aligning the first mating face 37 and second mating face 39 at the desired angle prior to allowing the two faces 37, 39 to mate.

The third mating face 41 is located on another side of the first end 19 of the handle 13 and is a relatively smooth surface. When the retractor blade 15 is positioned such that the second mating surface 39 is adjacent the third mating surface 41, and the projection 33 of the detent 29 is received in the groove 27, the retractor blade 15 is free to rotate relative to the handle 13 about the spigot 23 whilst being releasably secured thereto. In this arrangement the surgical instrument is in a first condition.

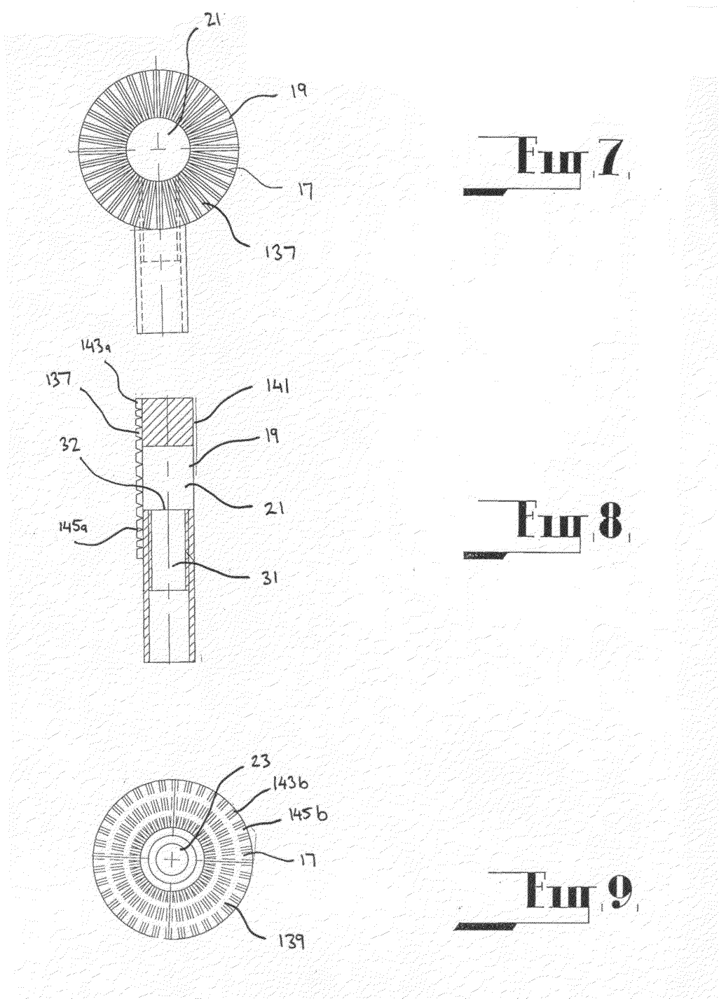

Referring to FIGS. 7, 8 and 9, a second embodiment of the invention is shown.

In this embodiment the first end 19 of the handle 13 incorporates a first mating face 137 having a plurality of serration like projections 143a. On a cooperating second mating face 139 located on the retractor blade 15 complementary serration like projections 143b are provided thereon such that when the first mating face 137 and second mating face 139 are brought into mating engagement the handle 13 will be locked relative to the retractor blade 15.

Similar to the first embodiment, the first end 19 of the handle 13 provides a third mating face 141 which provides a smooth face, such that when the third mating face 141 is adjacent the second mating face 139 of the retractor blade 15 the retractor blade 15 is free to rotate relative to the handle 13.

The attachment device provides three functions:
1. It allows the retractor blade to be located on the handle;
2. It provides means to releasably secure the retractor blade to the handle; and
3. It allows the retractor blade to be either locked/fixed, or be free to rotate relative to the handle.

These functions enable the surgical tool of the present invention to provide a simple and practical solution to the problems associated with the prior art, as previously stated. The surgical instrument enables a surgeon to have on hand a reduced number of handles and only the number of retractor blades which are required for the operation. In those cases where he/she requires a retractor blade which must be free to rotate the surgical instrument can be quickly assembled in a first condition. When he/she requires a surgical instrument which needs to be fixed at a relative angle, the surgical instrument is set up in the second condition. Furthermore, if the surgical instrument is set up at the incorrect angle it can be readily altered and then the retractor blade may easily be releasably secured to the handle. Also, if during surgery the surgeon requires a rotatable retractor blade which at the time is fixed relative to the handle, the surgeon only needs to remove the retractor blade, rotate it 180° and replace it on the handle such that the third mating face is adjacent the second mating face.

As there are no thumbscrews, clamps or other fastening means which require to be adjusted to secure or fix the retractor blade to the handle, the surgeon is able to easily adjust the retractor blade to the required condition and position it without being required to adjust screws or other small components.

The simple construction of the present invention also assists in the sterilisation process and minimises the chance of contamination between operations.

Modifications and variations such as would be apparent to the skilled addressee are considered to fall within the scope of the present invention.

Throughout the specification, unless the context requires otherwise, the word “comprise” or variations such as “comprises” or “comprising”, will be understood to imply the inclusion of a stated integer or group of integers but not the exclusion of any other integer or group of integers.

The claims defining the invention are as follows:

1. A surgical instrument comprising a handle, a retractor blade and an attachment device to releasably secure the retractor blade to a first end of the handle, whereby in a first condition the attachment device secures the blade to the handle such that the blade rotates relative to the handle, whilst in a second condition the attachment means secures the blade to the handle such that the blade is fixed relative thereto.

2. The surgical instrument according to claim 1 wherein the attachment device comprises a spigot which is received in an opening.

3. The surgical instrument according to claim 2 wherein the opening is in the first end of the handle, whilst the spigot extends from the retractor blade.

4. The surgical instrument according to claim 2 wherein the opening is in the retractor blade whilst the spigot extends from the first end of the handle.

5. The surgical instrument according to claim 2 wherein the attachment means comprises a detent retractably extending into the opening, the end of the detent being adapted to be received in an annular groove on the spigot.

6. The surgical instrument according to claim 5 wherein the detent is in the form of a projection biased outwardly from the handle into the opening.

7. The surgical instrument according to claim 1 wherein the attachment device provides a first mating face and a second mating face, the first and second mating faces being complementary to each other such that they are able to lock together.

8. The surgical instrument according to claim 7 wherein the first mating face and second mating face is in the form of one or more projections, slots or similar devices.

9. The surgical instrument according to claim 8 wherein the first mating face and second mating face can be locked at various angles such that the retractor blade is locked relative to the handle at various angles.

10. The surgical instrument according to claim 7 wherein the first mating face is provided by the handle.

11. The surgical instrument according to claim 7 wherein the second mating face is provided at an attachment end of the retractor blade.

12. The surgical instrument according to claim 1 wherein the handle has a third mating face.

13. The surgical instrument according to claim 12 wherein the third mating face is relative smooth such that when the third mating face is adjacent the second mating face the two surfaces cannot lock together, allowing the retractor blade to rotate relative to the handle whilst still being releasably secured thereto.
